(19) United States
(12) Patent Application Publication
(10) Pub. No.: US 2018/0353773 A1
(43) Pub. Date: Dec. 13, 2018

(54) TREATMENT PLANNING APPARATUS

(71) Applicant: HITACHI, LTD., Tokyo (JP)

(72) Inventors: Yusuke SAKAMOTO, Tokyo (JP); Kazushi HANAKAWA, Tokyo (JP)

(73) Assignee: HITACHI, LTD., Tokyo (JP)

(21) Appl. No.: 15/775,689

(22) PCT Filed: Nov. 17, 2015
(86) PCT No.: PCT/JP2015/082210

§ 371 Date: May 11, 2018

(51) Int. Cl. A61N 5/10

(52) U.S. Cl. CPC A61N 5/1039; A61N 2005/1074

(57) ABSTRACT

A treatment planning apparatus of the present invention comprises a processor configured to predict a degree of sharpness of an X-ray image which is acquired with assumed X-ray intensity and calculate position uncertainty of an affected area, calculate irradiation parameters of a therapeutic radiation based on the calculated position uncertainty of the affected area, calculate an X-ray irradiation amount with which a patient is irradiated by an X-ray imaging device with the assumed X-ray intensity and calculate a dose distribution of the therapeutic radiation which is irradiated to the patient using the calculated irradiation parameters of the therapeutic radiation and a display that displays the calculated X-ray irradiation amount and the calculated therapeutic radiation dose distribution.

### TECHNICAL FIELD

The present invention relates to a treatment planning apparatus for supporting a treatment plan in an image guided radiation therapy.

### BACKGROUND ART

Regarding a radiation therapy, Image Guided Radiation Therapy (IGRT), in which therapeutic radiation is irradiated while checking a position of an affected area in an image such as an X-ray image in order to accurately irradiate therapeutic radiation to an affected area is proposed (for example, Patent Document 1). In a case where an X-ray image is used as IGRT, a patient will be exposed to an X-ray for acquiring an image other than therapeutic radiation. It is preferable for the exposure to be as small as possible.

In Image Guided Radiation Therapy in which by using an X-ray imaging device, while checking a position of an organ, therapeutic radiation is irradiated, at a point when a treatment plan is made, an exposed dose caused by imaging an X-ray cannot be estimated, therefore, it is very inconvenient for a person who makes a treatment plan. For example, when the higher the intensity of an imaging X-ray is, the better the quality of an image is, therefore, the accuracy for estimating a position of an organ will be improved, however, there is trade off, that is, an exposed dose will also be increased. Consequently, when the treatment time can be estimated to some extent at a stage when a treatment plan is made, appropriate imaging X-ray intensity can be selected.

Further, in conventional radiation treatments, at a point when a treatment plan is made, the treatment time which is required cannot be estimated, therefore, it is inconvenient for a person who makes a treatment plan. For example, when the intensity of therapeutic radiation is higher, performing the treatment can be completed in a shorter time. However, there is trade off, that is, the lower the intensity of therapeutic radiation is, irradiation with higher accuracy can be performed. Consequently, when treatment time can be estimated to some extent at a stage when a treatment plan is made, appropriate intensity of a therapeutic beam can be selected.

### PRIOR ART DOCUMENT

### Patent Document

Patent Document 1 JP 2013-252420A

### Non Patent Document

Non Patent Document 1 Kawasaki et. al, “Optimal bladder volume in IMRT planning for prostate cancer” The journal of the Japanese Association of Radiological Technnologists 2015. Vol. 62 no. 751, pp 22-26

### SUMMARY OF THE INVENTION

### Problem to be Solved

As above mentioned, in IGRT in which an X-ray image is used, the higher the intensity of an imaging X-ray is, the better the quality of imaging is. However, there is trade off, that is, when the accuracy for estimating is improved, an exposed dose will also be increased. Consequently, a treatment planning apparatus, which can briefly determine the intensity of imaging X-ray by which an exposed dose can be made as small as possible, is desired.

The present invention aims to provide a treatment planning apparatus for supporting to make an appropriate treatment plan in which a user such as a doctor, etc. considers an exposed dose of radiation and treatment time.

### Means for Solving Problem

A treatment planning apparatus of the present invention comprises an X-ray imaging device for imaging an X-ray image of an affected area of a patient which is an irradiation objective, and is a treatment planning apparatus for making a treatment plan of a radiation treatment system in which therapeutic radiation is irradiated to the patient so as to treat the affected area based on an X-ray image which is imaged by the X-ray imaging device. The treatment planning apparatus of the present invention comprises an X-ray image position uncertainty calculation unit which calculates the position uncertainty of an affected area by predicting a degree of a sharpness of an X-ray image which is acquired by the assumed intensity of an X-ray, a therapeutic radiation irradiation parameter calculation unit which calculates irradiation parameters of the therapeutic radiation based on the position uncertainty of the affected area which is calculated by the X-ray image position uncertainty calculation unit, an X-ray irradiation amount calculation unit for calculating an X-ray irradiation amount for irradiating the patient by the X-ray imaging device with the X-ray intensity which is assumed, a therapeutic radiation dose distribution calculation unit which calculates a dose distribution of the therapeutic radiation for irradiating the patient by using the irradiation parameters of the therapeutic radiation which are calculated by the therapeutic radiation irradiation parameter calculation unit, and a display device for displaying the X-ray irradiation amount which is calculated by the X-ray irradiation amount calculation unit and the dose distribution of the therapeutic radiation which is calculated by the therapeutic radiation dose distribution calculation unit.

### Effects of Invention

The present invention can provide a treatment planning apparatus for supporting to make an appropriate treatment plan in which a user such as a doctor, etc. considers an exposed dose of radiation and a length of treatment time.

### BRIEF DESCRIPTION OF THE DRAWINGS

FIG. 1 is a block diagram showing the configuration of a treatment planning apparatus according to Embodiment 1 of the present invention.

FIG. 2 is a block diagram showing an example of hardware configuration of a treatment planning apparatus according to Embodiment 1 of the present invention.

FIG. 3 is a flowchart showing the operation of a treatment planning apparatus according to Embodiment 1 of the present invention.

FIG. 4 is a conceptual diagram showing the configuration of a particle beam treatment system as an example of a radiation treatment system including a treatment planning apparatus of the present invention.

FIG. 5 is a diagram showing the operation of a radiation treatment system including a treatment planning apparatus of the present invention.

FIG. 6 is a conceptual diagram showing the operation of a radiation treatment system including a treatment planning apparatus according to Embodiment 1 of the present invention.

FIG. 7 is a diagram showing an example of display by a treatment planning apparatus according to Embodiment 1 of the present invention.

FIG. 8 is a diagram showing another example of display by a treatment planning apparatus according to Embodiment 1 of the present invention.

FIG. 9 is a diagram showing another example of display by a treatment planning apparatus according to Embodiment 1 of the present invention.

### DETAILED DESCRIPTION OF THE PREFERRED EMBODIMENTS

### Embodiment 1

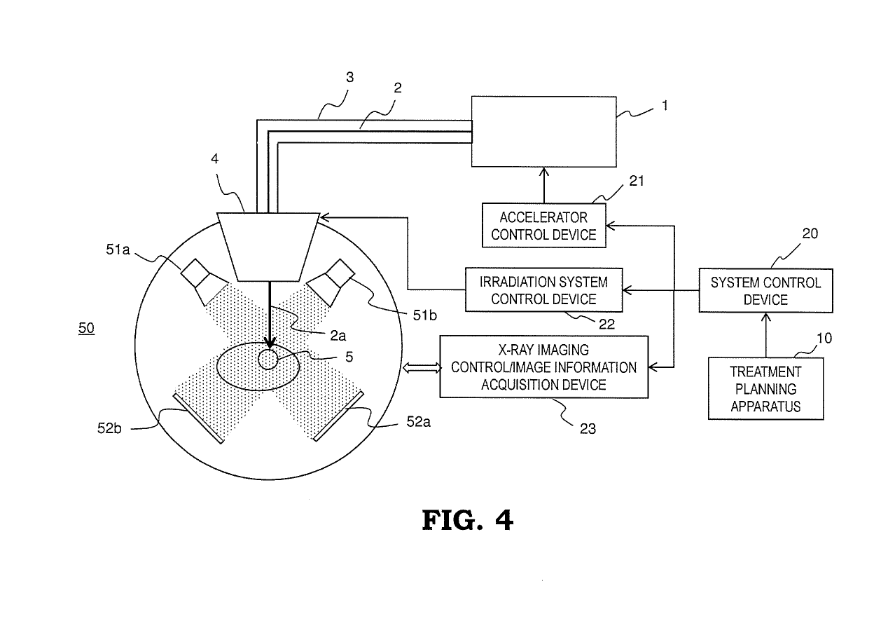

First, a particle beam treatment system will be described as an example of a radiation treatment system including a treatment planning apparatus of the present invention. FIG. 4 is a block diagram conceptually showing the configuration of one example of a particle beam treatment system including a treatment planning apparatus of the present invention. A particle beam 2, which is emitted as a high energy charged particle beam from an accelerator 1 which accelerates a charged particle beam, is passed through a vacuum duct 3 so as to be transmitted to an irradiation nozzle 4 which is provided in the downstream of the vacuum duct 3. Here, at a part of the vacuum duct 3 which is bent, a deflection electromagnet for changing the travelling direction of the particle beam 2 is provided, however, in FIG. 4, the schematic representation of a deflection electromagnet is omitted. The particle beam 2 is scanned in a two-dimensional direction which is perpendicular to the travelling direction of the particle beam 2 by a scanning electromagnet which is provided in the irradiation nozzle 4. A particle beam 2a which is scanned is irradiated to an affected area 5, of a patient who is lying down on a treatment bed, which is an irradiation objective. Various irradiation parameters in irradiating are set by a treatment planning apparatus 10, each parameters of the accelerator 1 and the irradiation nozzle 4 for irradiating with the irradiation parameter are set by a system control device 20 so as to be transmitted to an accelerator control device 21 and an irradiation system control device 22, and each command is outputted to each equipment of the accelerator 1 and the irradiation nozzle 4, respectively.

On the other hand, for example, in order to check the movement of the affected area 5 which is an irradiation objective by acquiring an X-ray image, an X-ray imaging device 50 comprising X-ray tubes 51a, 51b and flat panel detectors (FPD) 52a, 52b is provided. An X-ray which is emitted from the X-ray tube 51a is detected by the FPD 52a, and an X-ray which is emitted from the X-ray tube 51b is detected by the FPD 52b, respectively. The X-ray tubes 51a, 51b and the FPDs 52a, 52b are controlled by an X-ray imaging control/image information acquisition device 23 so as to acquire X-ray image information.

A method for treating an affected area such as a tumor by irradiating a particle beam which is therapeutic radiation to the affected area 5 of a patient according to the above-mentioned particle beam treatment system will be briefly described. First, in the treatment planning apparatus 10, an irradiation dose to be irradiated to the affected area 5 will be determined. The irradiation dose will be determined as a three-dimensional distribution in accordance with a shape of the affected area 5, that is, as an irradiation dose distribution. When an irradiation dose distribution is determined, in the treatment planning apparatus 10, an irradiation parameter, which is a set of various parameters of the accelerator 1 and the irradiation nozzle 4 for giving the irradiation dose distribution to the affected area, can be determined. However, a set of an irradiation parameter cannot be uniquely determined based on the intensity of a particle beam, a diameter of a beam, etc. Therefore, an irradiation parameter, which is considered as an appropriate parameter by a user such as a doctor, will be determined.

In a case of a particle beam treatment, irradiation of a particle beam to an affected area will be performed once a day, divided into times of several tens. On the date of performing irradiation, for example, the position of a patient who is lying on a treatment bed will be determined by controlling the position of the treatment bed so as for a patient's isocenter which is set in advance in an image of the affected area 5 of a patient which is acquired by an X-ray imaging control/image information acquisition device 23 to confirm to an isocenter of equipment which is determined by the irradiation nozzle 4. When the position determination is completed, each equipment is controlled by predetermined parameter of the accelerator 1 and the irradiation nozzle 4 via the accelerator control device 21 and the irradiation system control device 22 so as to irradiate a particle beam to the affected area 5. At this time, an X-ray image is acquired by the X-ray imaging control/image information acquisition device 23 in real time, while monitoring the position and movement of the affected area 5, for example, in the phase in which an amount of movement of the affected area 5 in the respiration phase is small, a particle beam is irradiated to the affected area 5. When performing irradiation of an irradiation dose to the affected area, which is planned for the day, is completed, performing of irradiation of that day will be terminated.

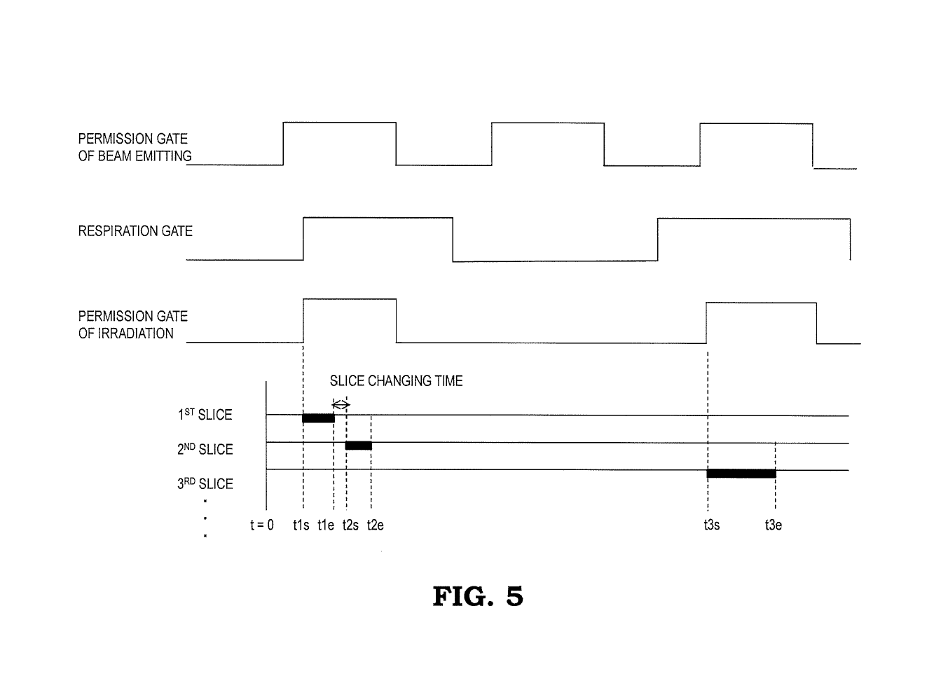

In a case where the accelerator 1 is a synchrotron, regarding a particle beam, only an amount of charged particles which are accumulated in the accelerator can be taken out. Therefore, one operation cycle of an accelerator consists of acceleration, beam emitting and deceleration; an accelerator repeats the operation cycle, and irradiation can be performed only at beam emitting. In many cases, with regard to one irradiation for one patient, a plurality of operation cycles are required. FIG. 5 shows the above mentioned operation cycle. FIG. 5 shows an irradiation method so called a scanning irradiation method. A scanning irradiation method is a method in which a particle beam which is bundle of charged particles is irradiated on an objective to be irradiated while the particle beam is scanned in a two-dimensional direction which is perpendicular to the travelling direction of the beam by a scanning electromagnet which is provided in the irradiation nozzle 4. An irradiation position in a travelling direction of a beam, that is, a depth direction is determined by energy of a charged particle to be irradiated, therefore, by changing energy of a charged particle, an irradiation position in a depth direction can be changed. As above mentioned, a three dimensional position to be irradiated is controlled to irradiate a particle beam. Generally, in a scanning irradiation method, a dose is controlled at every irradiation position so as to irradiate a particle beam. In a scanning irradiation method, a position of an affected area in a depth direction according to energy of a charged particle is irradiated, therefore, by scanning a particle beam in a two dimensional direction with a scanning electromagnet per energy of charged particles so as to irradiate, the layered irradiation area will be irradiated in return every time when energy is changed. The above mentioned layered irradiation area will be called as a slice.

FIG. 5 is a diagram showing the operation of a radiation treatment system in which by changing energy of charged particles of a particle beam which is irradiated to the affected area 5, per slice of the affected area 5 is irradiated. A horizontal axis of FIG. 5 indicates the time. The time when a beam of an accelerator can be emitted is predetermined time per one operation cycle as above mentioned. By irradiating a particle beam at the time when the movement of the affected area caused by patient's respiration is decreased, position accuracy of irradiation can be improved, therefore, it is intended to irradiate the affected area at the above mentioned time. The above mentioned time will be called as a respiration gate. The time when beam can be emitted and the time of a respiration gate is overlapped is the time when a particle beam can be irradiated to a patient. As shown in FIG. 5, at the time of t1s, performing irradiation to a first slice starts, irradiation is performed while scanning all positions of the first slice so as to terminate performing irradiation, at the time of t2s after the changing time for changing energy of a particle beam in order to irradiate following second slice, performing irradiation to the second slice starts. Irradiation is performed while scanning all positions of the second slice so as to terminate performing irradiation to the second slice. At this point, remaining time when irradiation can be performed for a patient is less, therefore, performing irradiation to following third slice starts at the time of t3s, that is, next irradiation-able time for a patient, and irradiation is performed while scanning all positions of the third slice so as to terminate performing irradiation to the third slice.

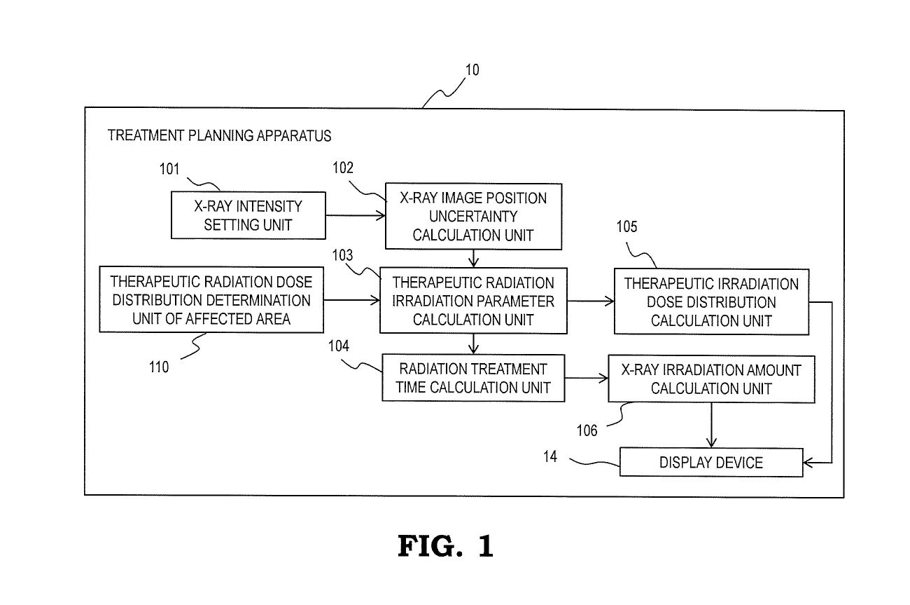

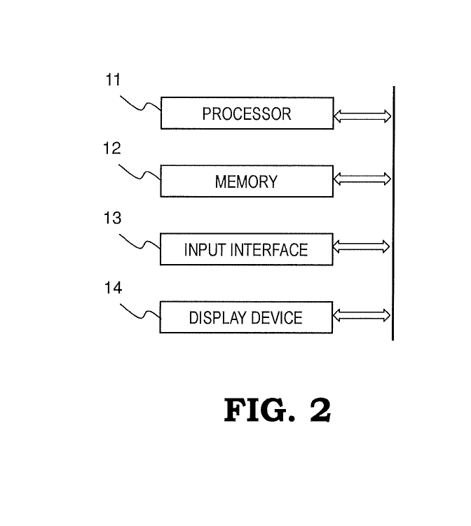

The present invention provides a treatment planning apparatus for supporting to make a treatment plan in order to reliably irradiate to the affected area 5 with less exposure by predicting exposure to an X-ray irradiation, exposure to a particle beam irradiation and especially exposure to an area other than the affected area 5 by performing the above mentioned irradiation. FIG. 1 is a block diagram showing an essential part of a treatment planning apparatus according to Embodiment 1 of the present invention. The state of actual irradiation which is described in the above can be predicted by the treatment planning apparatus 10 in advance. By performing the above mentioned prediction, the X-ray irradiation time for acquiring an X-ray image which observes and monitors the movement of the affected area 5 in irradiating can be predicted. Further, the treatment planning apparatus 10 can be realized by general calculators including a processor 11, a memory 12, an input interface 13 such as a keyboard and a touch panel, a display device 14 as an output interface as shown in FIG. 2.

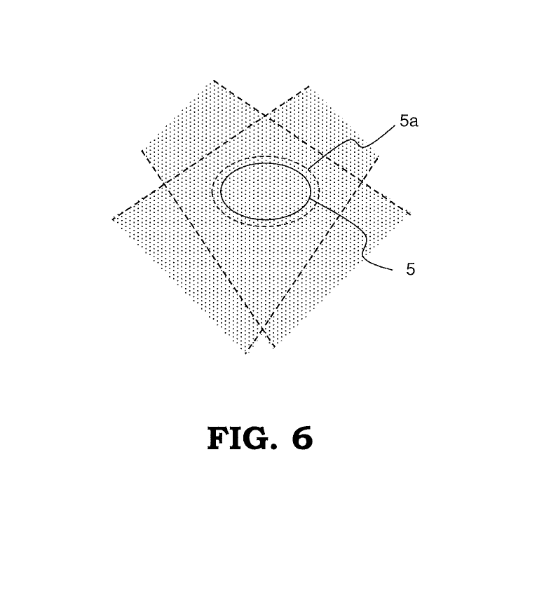

Regarding X-ray intensity for acquiring an X-ray image, from a view point of exposure dose of a patient, low intensity is preferable. However, when X-ray intensity is weak, a degree of sharpness of an image which is acquired by FPD is low, therefore, the position uncertainty of an affected area is high. As above mentioned, the relationship between the X-ray intensity and the position uncertainty is trade off. When the X-ray intensity is weak, a dose of an X-ray is low, therefore, exposure dose of a patient is also low. However, when the position uncertainty is high, an outline of an affected area is blurry and indistinct, therefore, it is necessary to make the margin of particle beam irradiation to be large. That is, as shown in FIG. 6, in order to surely give a dose of a particle beam to the affected area 5, it is necessary to irradiate to an area which is larger than the outline of the affected area, for example, an area 5a which is indicated with the broken line. In a case where an area which is larger than the outline of the affected area is irradiated, normal tissue in a periphery of the affected area is also irradiated by a particle beam. On the other hand, when the X-ray intensity is strong, a position uncertainty is low, that is, a position of an affected area can be clearly discriminated, therefore, the margin of particle beam irradiation can be set to be low. Consequently, an irradiation amount to a periphery of the affected area can be made to be small. As above mentioned, the relationship between an X-ray irradiation amount and an irradiation dose of a particle beam which is irradiated to a part other than an affected area is trade off.

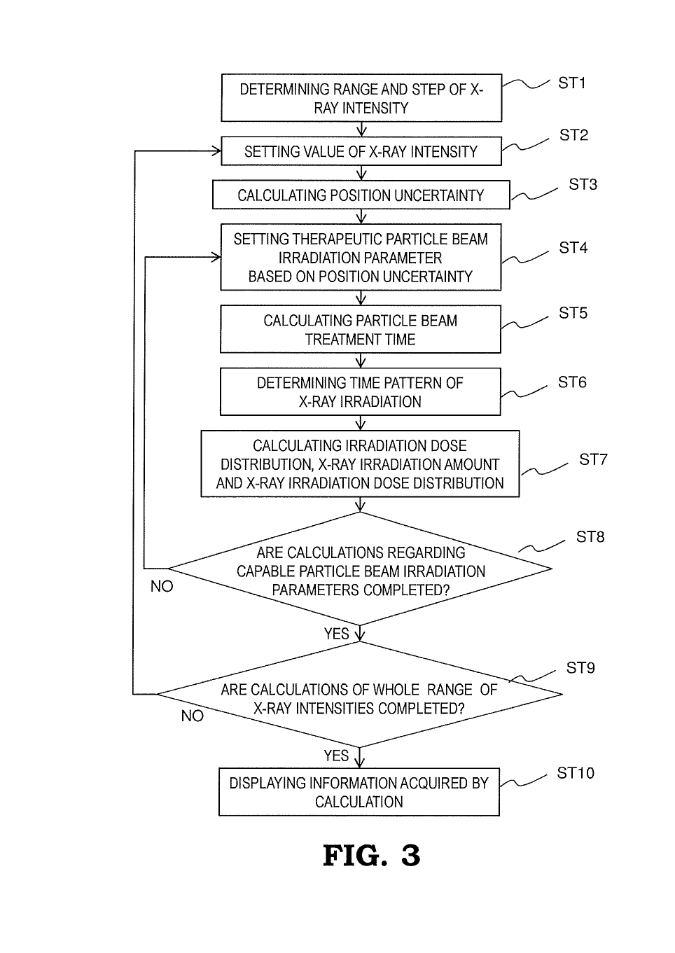

The present invention provides a treatment planning apparatus for supporting to determine an intensity of an X-ray in making a treatment plan by presenting an X-ray irradiation amount and an irradiation dose of a particle beam which is irradiated to a part other than an affected area, which is in the trade off relationship. In the following, the treatment planning apparatus 10 according to Embodiment 1 of the present invention will be described referring a block diagram of FIG. 1 and a flow chart of FIG. 3. As above mentioned, the treatment planning apparatus 10 is realized by a calculator shown in FIG. 2, and following each part and each step will be realized by performing a program which is stored in a memory 12 with a processor 11.

First, in an X-ray intensity setting unit 101, a range of X-ray intensity and a step of X-ray intensity will be determined (ST1). Next, a value of X-ray intensity is set to be the weakest value of X-ray intensity (ST2). In an X-ray image position uncertainty calculation unit 102, by predicting an X-ray image which is acquired by the value of X-ray intensity which is set, the position uncertainty will be calculated (ST3).

Next, based on the position uncertainty which is calculated by the X-ray image position uncertainty calculation unit 102, by a therapeutic radiation irradiation parameter calculation unit 103, therapeutic particle beam irradiation parameters are set so as to satisfy a dose distribution of an affected area which is determined by a therapeutic radiation dose distribution determination unit of an affected area 110 (ST4). Time pattern of particle beam irradiation and treatment time by irradiation parameters which are set by the therapeutic radiation irradiation parameter calculation unit 103 will be obtained by performing prediction calculation in a radiation treatment time calculation unit 104 (ST5). Here, in a case where radiation is irradiated dividedly to a plurality of areas to be irradiated such as scanning irradiation of a particle beam, a layer-stacking conformal irradiation, etc., by considering time which will be required when an irradiation area (slice) is switched, necessary number of gate where a beam can be emitted and that of gate where a particle beam can be irradiated to a patient will be estimated so as to assume treatment time which will be required. By reflecting the switching time of area to be irradiated, treatment time which will be required can be known.

Regarding performing irradiation from a plurality of gantry angles such as multi-port irradiation, IMRT, IMPT, etc., in a case where performing irradiation from one angle is completed, irradiation is consequently performed from following second angle while a patient is kept to lie down in a treatment bed, without re-determining the positioning, by considering time which will be required for changing a gantry angle, necessary number of gate where a beam can be emitted and that of gate where beam can be irradiated to a patient will be estimated so as to assume treatment time which will be required. By reflecting time which is required for gantry rotation, treatment time which will be required can be known.

Further, in a case where respiration synchronism irradiation is performed to an organ with respiratory movement, a respiration coaching system for stabilizing a respiration cycle can be introduced. By introducing the respiration coaching system, a respiration cycle will be stable, as a result, the accuracy for predicting treatment time can be expected.

Next, based on a pattern of time of particle beam irradiation which is obtained by performing a prediction calculation, a time pattern of an X-ray irradiation will be determined (ST6). By using each parameter which is determined in the above mentioned and predicted time, a dose distribution of a particle beam which is irradiated therapeutically, that is, a therapeutic radiation irradiation dose distribution will be calculated by a therapeutic radiation dose distribution calculation unit 105 and an X-ray irradiation amount which is irradiated for imaging will be calculated by an X-ray irradiation amount calculation unit 106 (ST7). By the X-ray irradiation amount calculation unit 106, an X-ray irradiation dose distribution may be calculated together with an X-ray irradiation amount. In a case where another particle beam irradiation parameter can be set based on the position uncertainty (ST8 NO), a procedure will be returned to step ST4, another particle beam irradiation parameter in which intensity of a particle beam, dose limiting condition, etc. is changed will be set. When calculation regarding capable particle beam irradiation parameters based on the position uncertainty are completed (ST8 YES and ST9 NO), a procedure will be returned to step ST2, next X-ray intensity will be set, and regarding the X-ray intensity, the position uncertainty will be calculated by predicting an X-ray image to be acquired (ST3). When calculations of whole range of X-ray intensities which are determined by ST1 are completed (ST9 YES), various information including X-ray irradiation amount which is calculated and a therapeutic radiation dose distribution will be displayed in a display device 14 (ST 10). At this time, predicted treatment time may also be displayed. A user such as a doctor, etc., will check the result which is displayed, and will determine appropriate intensity of an X-ray and a particle beam irradiation parameter, and a treatment planning apparatus will make a treatment plan based on determined X-ray intensity and particle beam irradiation parameter.

Regarding display in a display device, not only an X-ray irradiation amount which is a value of exposed dose but also a three dimensional distribution of an X-ray irradiation amount which is calculated by the X-ray irradiation amount calculation unit 106 may be displayed. Further, a three dimensional distribution of an X-ray irradiation amount may be displayed side by side with a therapeutic radiation dose distribution which is calculated by a treatment plan. Alternatively, a combined distribution of a therapeutic radiation dose distribution and an X-ray irradiation dose distribution for imaging will be displayed. By doing the above mentioned, a person who makes a treatment plan can visually know the effect which is given to a patient with exposed dose.

By doing the above mentioned, based on information of radiation treatment plan (a therapeutic beam amount) and information of therapeutic beam generating apparatus (intensity of therapeutic beam and a cycle of a period with which a beam can be emitted), necessary number of gate where a beam can be emitted will be estimated, further, based on information regarding X-ray intensity of an X-ray imaging device (a value of exposed dose per one time of imaging and frequency of imaging), an exposed dose of imaging X-ray (an X-ray irradiation amount) will be estimated so as to display in the display device 14. An X-ray irradiation amount may be an integrated value of dose of whole of patient or a value of dose at a local point of representative point (such as isocenter). At a stage of making a treatment plan, a person who makes a treatment plan can know a predicted value of X-ray exposure dose.

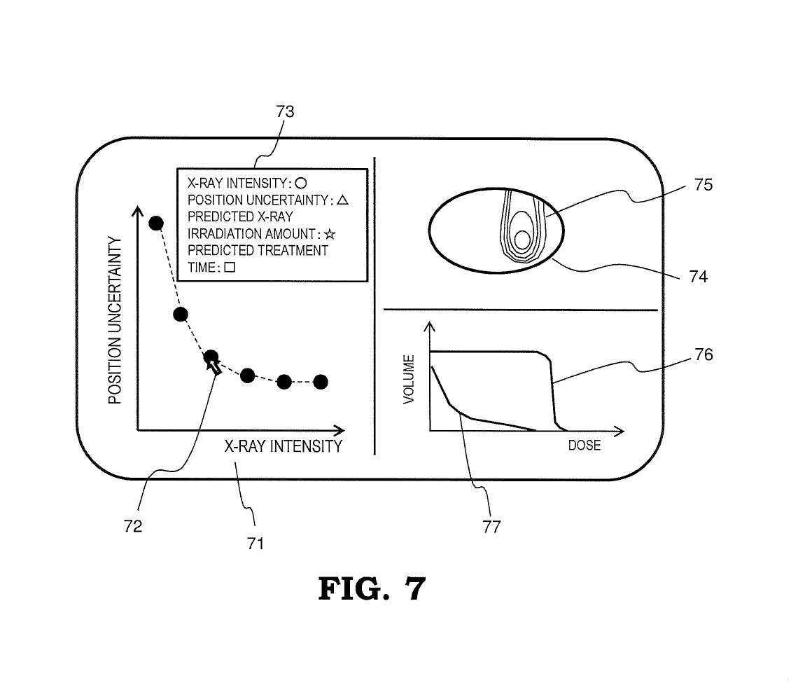

Further, with regard to a plurality of values of X-ray intensity, expected position uncertainty and a predicted amount of X-ray irradiation may be displayed, respectively. For example, a graph where the X-ray intensity is plotted at a horizontal axis and the position uncertainty is plotted at a vertical axis may be displayed, and when a plot point is selected, the predicted amount of X-ray irradiation and predicted treatment time at the plot point may be displayed. In FIG. 7 to FIG. 9, an example of display image which is displayed in the display device 14 will be shown.

FIG. 7 shown an example of typical display in a display device. A graph 71, where the X-ray intensity is displayed at a horizontal axis and the position uncertainty is displayed at a vertical axis is displayed, is displayed, and a point responding to whole of a treatment plan which is made is plotted. When a user selects one of points which are plotted, for example, the selected point is designated by using a mouse button 72, etc., summary information will be displayed in a summary display window 73 in the displayed image. Here, the summary information includes the X-ray intensity, position uncertainty, a predicted X-ray irradiation amount, a predicted treatment time, etc.

Further, by overlapping with a patient CT 74, a therapeutic dose distribution 75 which is planned will be displayed. At the same time, Dose Volume Histogram 76 (DVH) with regard to a target (PVT: Planning Target Volume) and DVH 77 with regard to an organ at risk (OAR) will be displayed.

Here, as one example, regarding a patient CT, information of a cross section taken out from a three dimensional CT information is displayed, however, three sections responding to each direction in a three dimension may be displayed side by side. Further, here, as one example, DVH having one PTV and one OAR is displayed, however, in a case where there are a plurality of OAR, a plurality of OAR may be displayed. Further, in this example, whole information is displayed in one display device, however, whole information may be divided so as to display in a plurality of display devices. Alternately, whole information may be displayed in one display device by switching images.

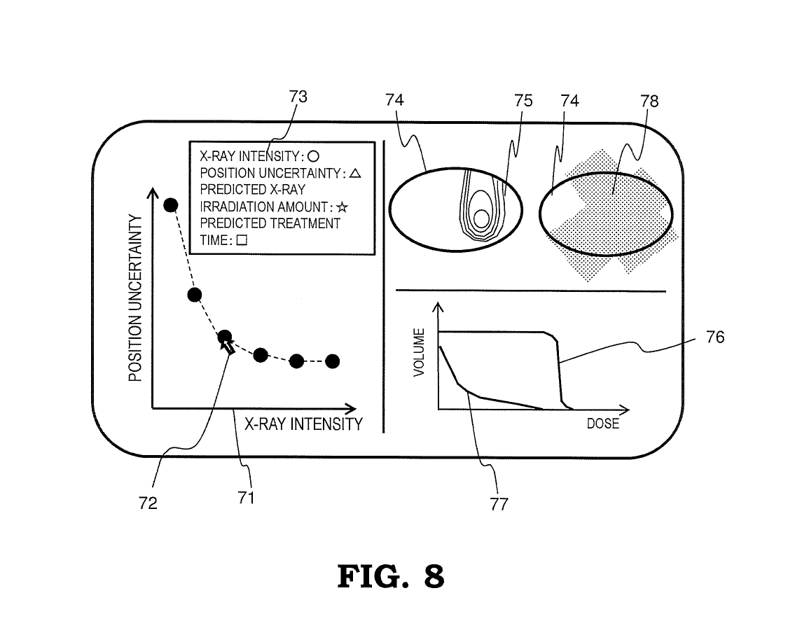

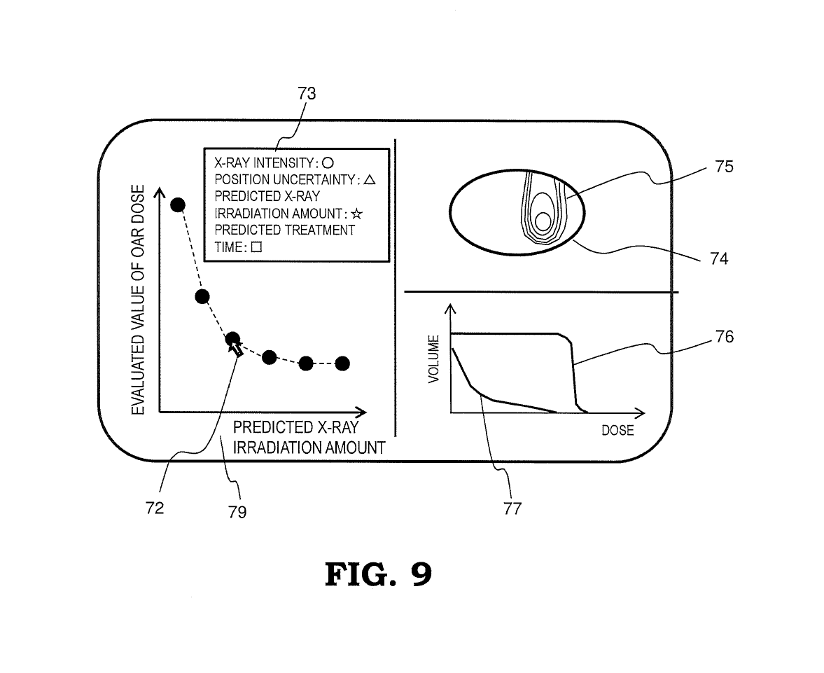

FIG. 8 shows an example of display where more information is shown than that in FIG. 7. Not only a therapeutic dose distribution 75 but also an irradiation dose distribution of an X-ray for imaging 78 will be displayed. Further, FIG. 9 shows an alternative example of graph to be displayed. As a graph which is displayed in the left side of FIG. 7 and FIG. 8, a graph 79 shown in FIG. 9, that is, a graph where a predicted X-ray irradiation amount is plotted at a horizontal axis and an evaluated value of OAR dose is plotted at a vertical axis, respectively, may be displayed.

Here, an evaluated value of OAR dose is a value of V20, etc. V20 indicates a value, indicating with percent, of the ratio of volume at a part where the dose exceeds 20 Gy amount to OAR volume, and is an indicator which is generally used for determining dose limitation. In making a treatment plan, it is important for the value to be smaller than a reference value which is determined per a treatment facility. For example, in Non Patent Document 1, regarding treatment of a prostate cancer, a reference example, in which V67.1 in a rectal wall is less than 25% and V42.0 is less than 40%, is shown. By displaying the above mentioned clinical parameter in a display device, it can be expected for a user having a clinical view point to understand the trade-off relationship more easily.

By displaying the above mentioned information, the trade-off relationship between exposure dose and the accuracy of position estimation will be clear, and consequently, the most appropriate value of X-ray intensity can be easily selected.

Further, with regard to a plurality of values of imaging X-ray intensity, based on the position uncertainty of an affected part to be predicted, each of target margin will be determined, by using the target margin, a treatment plan is made, and each of result of treatment plan (dose distribution, DVH, etc.) may be displayed side by side.

### DESCRIPTION OF REFERENCE CHARACTERS

1: treatment planning apparatus
14 display device
50: X-ray imaging device
102: X-ray image position uncertainty calculation unit
103: therapeutic radiation irradiation parameter calculation unit
105: therapeutic irradiation dose distribution calculation unit
106: X-ray irradiation amount calculation unit

1. A treatment planning apparatus, for making a treatment plan of a radiation treatment system in which based on an X-ray image which is imaged by an X-ray imaging device which images an X-ray image of an affected area of a patient which is an irradiation objective, therapeutic radiation is irradiated to the patient so as to treat the affected area, comprising
a processor configured to assume X-ray intensity of the X-ray imaging device, predict a degree of sharpness of an X-ray image which is acquired by the X-ray intensity which is assumed and calculate position uncertainty of the affected area by the X-ray image;
calculate irradiation parameters of the therapeutic radiation based on the position uncertainty of the affected area which is calculated;
calculate an X-ray irradiation amount for irradiating the patient by the X-ray imaging device with the X-ray intensity which is assumed;
calculate a dose distribution of the therapeutic radiation for irradiating the patient by using the irradiation parameters of the therapeutic radiation which is calculated; and
a display device for displaying the X-ray irradiation amount which is calculated and the therapeutic radiation dose distribution which is calculated.

2. The treatment planning apparatus as claimed in claim 1 wherein the X-ray intensity which is assumed is a plurality of X-ray intensity, the processor is configured to calculate the position uncertainty of the affected area corresponding to each X-ray intensity of the plurality of X-ray intensity and calculate the irradiation parameters of the therapeutic radiation based on the position uncertainty of the affected area corresponding to each X-ray intensity of the plurality of X-ray intensity, and the display device displays the X-ray irradiation amount and the therapeutic radiation dose distribution regarding each of the plurality of X-ray intensity.

3. The treatment planning apparatus as claimed in claim 1, wherein the processor is configured to calculate an X-ray irradiation amount distribution in addition to the X-ray irradiation amount and the display device displays the X-ray irradiation dose distribution as the X-ray irradiation amount to be displayed.

4. The treatment planning apparatus as claimed in claim 2, wherein the processor is configured to calculate an X-ray irradiation amount distribution in addition to the X-ray irradiation amount and the display device displays the X-ray irradiation dose distribution as the X-ray irradiation amount to be displayed.

5. A radiation treatment system comprising the treatment planning apparatus as claimed in claim 1.
